(19) United States
(12) Patent Application Publication
(10) Pub. No.: US 2012/0277517 A1
(43) Pub. Date: Nov. 1, 2012

(54) Formulation and Methods for Enhanced Interventional Image-Guided Therapy of Cancer

(75) Inventors: Robert Ivkov, Ellicott City, MD (US); Eleni Liapi, Baltimore, MD (US)

(73) Assignee: The Johns Hopkins University, Baltimore, MD (US)

(21) Appl. No.: 13/442,305

(22) Filed: Apr. 9, 2012

Related U.S. Application Data
(60) Provisional application No. 61/473,496, filed on Apr. 8, 2011.
(60) Provisional application No. 61/473,504, filed on Apr. 8, 2011.

(51) Int. Cl. A61M 37/00; A61P 35/00; A61K 51/00; A61K 9/10; A61K 33/26

(52) U.S. Cl. 600/2; 424/490; 424/646; 424/1.11; 424/647; 424/493; 600/12

(57) ABSTRACT

An embodiment in accordance with the present invention provides a thermo-chemoembolization formulation and method for enhanced interventional image-guided therapy for cancer. The T-C formulation includes magnetic iron oxide nano-particles (MIONs) that heat when exposed to an alternating magnetic field (AMF), a liquid tumorphilic drug carrier that enhances tumor retention of the T-C formulation, and a chemotherapeutic or radiotherapeutic agent. The T-C formulation enhances delivery of heat and chemo- or radio-therapeutic agents with hyperthermia produced by magnetic nanoparticles to improve therapeutic outcomes. The magnetic nanoparticles and tumorphilic drug carrier also allow for multimodal image-guided monitoring of treatment and patient follow-up. The method for enhanced interventional image-guided therapy for cancer includes using an AMF to heat the T-C formulation and activate the thermotherapy.

### CROSS REFERENCE TO RELATED APPLICATIONS

This application claims the benefit of U.S. Provisional Patent Application No. 61/473,496, filed Apr. 8, 2011, and U.S. Provisional Patent Application No. 61/473,504, filed Apr. 8, 2011, both of which are incorporated by reference herein, in their entirety.

### FIELD OF THE INVENTION

The present invention relates generally to cancer treatment. More particularly, the present invention relates to a formulation and method for hyperthermia treatment of cancer.

### BACKGROUND OF THE INVENTION

Cancer is the leading cause of mortality and morbidity and, therefore, continues to be a global health concern. Effective treatment of the local or definitive, and locally recurrent disease remains a challenge in clinical settings, primarily to reduce therapy-related morbidity. For most patients only non-surgical palliative treatments are offered due to advanced disease at presentation, or other complicating factors. Interventional image-guided techniques for cancer therapy are often used as palliative treatment for many solid tumor cancers that are contraindicated for surgery because of their advanced stage at diagnosis or proximity to sensitive organs or structures.

Chemo-embolization and trans-arterial chemoembolization (TACE) have demonstrated modest success in a palliative setting for unresectable cancers of the liver and kidneys. However, significant improvements in survival outcome have not generally been observed with these procedures. Further, local recurrent disease tends to be resistant to chemotherapeutic agents because these comprise standard of care use for therapy following diagnosis. On the other hand, hyperthermia with either chemo- or radio-therapies has demonstrated improved response with survival benefit for many cancers and recurrent disease, largely because heat has a profound effect on proteins involved in repair mechanisms.

Ablative heating, one common interventional technique offers palliation, but is typically accompanied by significant morbidity because nearby healthy tissues are often extensively damaged. Hyperthermia, or heating cells to a temperature of between 39° C. and 49° C., is toxic to cancer and also sensitizes cancer cells to chemotherapy and radiation. However, lack of precision and tendency to “overtreat” are among the drawbacks that limit the utility and clinical adoption of heat-based techniques.

It would therefore be advantageous to provide a formulation and method that provides better precision of delivery of hyperthermia treatment in an interventional setting with fine control of dose-deposition along with compatibility.

### SUMMARY OF THE INVENTION

The foregoing needs are met, to a great extent, by the present invention, wherein in one aspect, a thermo-chemoembolization (T-C) formulation for treating a tumor in a subject, includes a tumorphilic carrier fluid that selectively accumulates in or near tumor cells to enhance retention of the compound within the tumor, such that the T-C formulation is deliverable either intra-tumorally or intra-arterially. The formulation can also include a biocompatible suspension of magnetic iron oxide nanoparticles (MIONs) having a magnetic iron oxide core that produces at least 50 Watts of heat per gram iron when subjected to an alternating magnetic field having a frequency between 100 kHz (1×10³ Hz) and 1 MHz (1×10⁶) and a peak-to-peak amplitude of between 5 kA/m and 100 kA/m. The magnetic iron oxide core is surrounded by a coating. Additionally, the formula can include an emulsifying agent.

In accordance with another aspect of the present invention, the tumorphilic carrier fluid can take the form of ethiodized oil. The magnetic iron oxide core includes at least one of the group of γFe₂O₃ and Fe₃O₄. The core can be made of at least one crystal of γFe₂O₃ and/or Fe₃O₄. The coating can take the form of at least one of the group of a biocompatible polymer and a biocompatible surfactant. If a biocompatible polymer is used, it can include at least one of the group of starch, dextran, and polyethylene glycol. Alternately, if a biocompatible surfactant is used, it can take the form of at least one of the group of citric acid, phospholipid, and polysorbate. The emulsifying agent can be at least one of the group of chelators such as a polyamino carboxylic acid or a macrocycle chelators, biocompatible surfactants, and polysorbate, and if a polyamino carboxylic acid is used, it can take the form of at least ethylenediaminetetraacetic acid (EDTA); and, if a macrocycle chelator is used, it can take the form of at least one of the group of 1,4,7,10-tetraazacyclododecane-1,4,7-tetraacetic acid (DOTA).

According to another aspect of the present invention, the formulation can include an anti-cancer agent comprising of one of the group of at least one of chemotherapy agent and a radiotherapy agent. If a chemotherapy agent is used, it can take the form of at least one of the group of of cisplatin, carboplatin, cyclophosphamide, docetaxel, doxorubicin, gemcitabine, ifosfamide, irinotecan, melphalan, mitomycin, mitoxantrone, oxaliplatin, topotecan, vinorelbine, tamoxifen, and paclitaxol. Alternately, if a radiotherapy agent is used, it can be one of the group of ⁹⁰Y, ¹²⁵I, ¹³¹I, ⁶⁰Co, ¹⁹²Ir, ⁸⁹Sr, ¹⁵³Sm, ¹⁸⁶Re, and ^{99m}Tc. A radiolabeling agent can also be included and can take the form of at least one of the group of ¹⁸F, ⁶⁴Cu, and ¹¹¹In.

In accordance with yet another aspect of the present invention, a method for treating a tumor in a subject includes administering to the subject a tumorphilic formulation comprising a biocompatible suspension of magnetic iron nanoparticles (MIONs) having a magnetic iron oxide core surrounded by a coating. The method can also include positioning the subject in an alternating magnetic field (AMF) and applying the AMF to inductively heat the MIONs such that the MIONs increase in temperature. Additionally, the method can include administering an anti-cancer agent to the subject.

According to still another aspect of the present invention, the method can further include generating the AMF with a solenoid, and reducing field inhomogenities with high magnetic permeability capping rings positioned on the solenoid. The method can also include tuning the AMF to a particular frequency in its range.

According to another aspect of the present invention, a method for treating a tumor in a subject includes delivering via a catheter to an artery adjacent to a tumor a tumorphilic thermo-chemoembolization (T-C) formulation comprising a biocompatible suspension of magnetic iron nanoparticles (MIONs) having a magnetic iron oxide core surrounded by a coating. The method can also include positioning the subject in an alternating magnetic field (AMF). Further, the method can include applying the AMF to heat the MIONs such that the MIONs increase in temperature, and administering an anti-cancer agent to the subject.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a graph of the diameter of the starch-BNF nanoparticles in phosphate buffered saline (PBS) and also with poly D-lysine, a biocompatibilizing agent.

FIG. 2 illustrates a transmission electron microscopy image from a starch-BNF nanoparticle.

FIG. 3 illustrates a graph of the diameter of the dextran-coated nanoparticles stabilized with citrate and suspended in water.

FIG. 4 illustrates a transmission electron microscopy image from a dextran-coated nanoparticle.

FIG. 5 illustrates a graph of the temperature over time for a sample of starch-BNF nanoparticles activated by AMF.

FIG. 6 illustrates a graph of the heating efficiency for the starch-BNF nanoparticles.

FIG. 7 illustrates a perspective view of an AMF device according to an embodiment of the invention.

FIG. 8 illustrates an AMF system according to an embodiment of the invention.

FIG. 9 illustrates a graph depicting the in-vitro heating capacity of four mixtures with a constant volume of 3 ml containing various concentrations of iron and ethiodized oil.

FIG. 10 illustrates a mouse used in an experiment using a T-C formulation according to an embodiment of the invention.

FIG. 11 illustrates the results of a biolluminence imaging (BLI) at 12 hours following injection and before AMF hyperthermia treatment and at 24 hours after AMF hyperthermia treatment.

FIG. 12 illustrates temperature readings over time for a mouse in an experiment using a T-C formulation according to an embodiment of the invention.

FIGS. 13A-13C illustrate histopathological images of the Prussian Blue stained liver VX2 tumor slides at 7 days following treatment for the New Zealand white rabbits according to an embodiment of the invention.

FIGS. 14A-14D show images of the New Zealand white rabbit liver over the experiment according to an embodiment of the invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a thermo-chemoembolization (T-C) formulation and method for enhanced interventional image-guided therapy for cancer. The T-C formulation includes magnetic iron oxide nano-particles (MIONs) that heat when exposed to an alternating magnetic field (AMF), a liquid tumorphilic drug carrier that enhances tumor retention of the T-C formulation, and a chemotherapeutic or radiotherapeutic agent. The T-C formulation enhances delivery of heat and chemo- or radio-therapeutic agents with hyperthermia produced by magnetic nanoparticles to improve therapeutic outcomes. The magnetic nanoparticles and tumorphilic drug carrier also allow for multimodal image-guided monitoring of treatment and patient follow-up. The method for enhanced interventional image-guided therapy for cancer also includes using an AMF to heat the T-C formulation and activate the thermotherapy.

The main components of the T-C formulation to which any other chemotherapeutic or radioactive component may be added are: a) a tumorphilic carrier fluid such as ethiodized oil, b) a biocompatible suspension of magnetic iron oxide nanoparticles (MIONs), and c) an emulsifying or stabilizing agent. Each is described below in detail, as are other components and methods of applying heat to activate the T-C formulation's thermotherapeutic characteristics.

One component of the T-C formulation is ethiodized oil. Ethiodized oil is an iodinated derivative of poppy seed oil, containing ethyl esters of linoleic, oleic, palmitic and stearic acids, with iodine content of 38-40% w/v (as a naturally iodinated compound). Ethiodized oil is a tumorphilic drug carrier. For example, ethiodized oil, when injected into the artery that feeds primary or metastatic hepatic tumors, selectively accumulates in cancer cells. This phenomenon of uptake and retention has been used to deliver targeted therapies via the hepatic artery to primary and metastatic hepatic tumors. This can be accomplished either by formulating ethiodized oil to contain cytotoxic agents (such as doxorubicin) to give targeted chemotherapy, conjugating ethiodized oil to radioactive substances (such as ⁹⁰Y, ¹⁸⁸Re) or by radiolabelling some of the iodine in ethiodized oil with ¹³¹I to deliver targeted radiotherapy. Ethiodized oil alone shows no heating with alternating magnetic field (AMF) exposure, but absorbs heat from surrounding heated BNF particles. When heated, Ethiodized oil achieves greater tumor necrosis than its unheated counterpart.

The T-C formulation also includes magnetic iron oxide nanoparticles (MIONs) that include a magnetic iron oxide core that is coated with biocompatible polymer or biopolymer, such as starch or dextran, and suspended in an injectable aqueous formulation. The magnetic iron oxide core can take the form of γ-Fe₂O₃ or Fe₃O₄ or any other suitable ferrous compound. The MIONs exhibit a high degree of heatability, or specific loss power (SLP) when exposed to safe alternating magnetic fields (AMF). Indeed, the SLP can be greater than or equal to approximately 100 Watts/g iron. The MIONs are also biodegradable. Two exemplary MION formulations include a starch-coated bionized nanoferrite (starch-BNF) nanoparticle, and a dextran-coated nanoparticle. However, any suitable MION formulation known to one of skill in the art can be used.

The starch-BNF nanoparticles can be produced by precipitating ferric and ferrous sulfate salts from solution with high pH in a high-pressure-homogenization reaction vessel. The iron content can be approximately >70% w/w, with a total iron concentration of about 30 mg Fe/mL (42 mg particle/mL). However, any suitable iron concentration known to one of reasonable skill in the art can be used. The particles can then be suspended in sterile water to provide a stable biocompatible suspension.

For the dextran-coated nanoparticles, mixed phase magnetite-maghemite (Fe₃O₄/γ-Fe₂O₃) particles can be prepared in a small scale high-gravity controlled precipitation (HGCP) platform via co-precipitation method. An iron precursor solution can include anhydrous FeCl₃ and FeCl₂.4H₂O dissolved in water at elevated temperature. Under continuous flow of nitrogen gas, excess 25% ammonia solution can be added with vigorous stirring, and the reaction mixture turns black immediately. Citric acid solution can then be added to form a stable suspension of the nanoparticles. The nanoparticles can be separated by centrifugation and washed several times with water and acetone to achieve dispersion at pH 6-8. The trace of acetone can be removed under reduced pressure at 60° C. for 15 minutes before the nanoparticles are treated hydrothermally at high temperature for several hours. The final citrate-stabilized dextran-coated nanoparticles can then be washed and resuspended in sterile water.

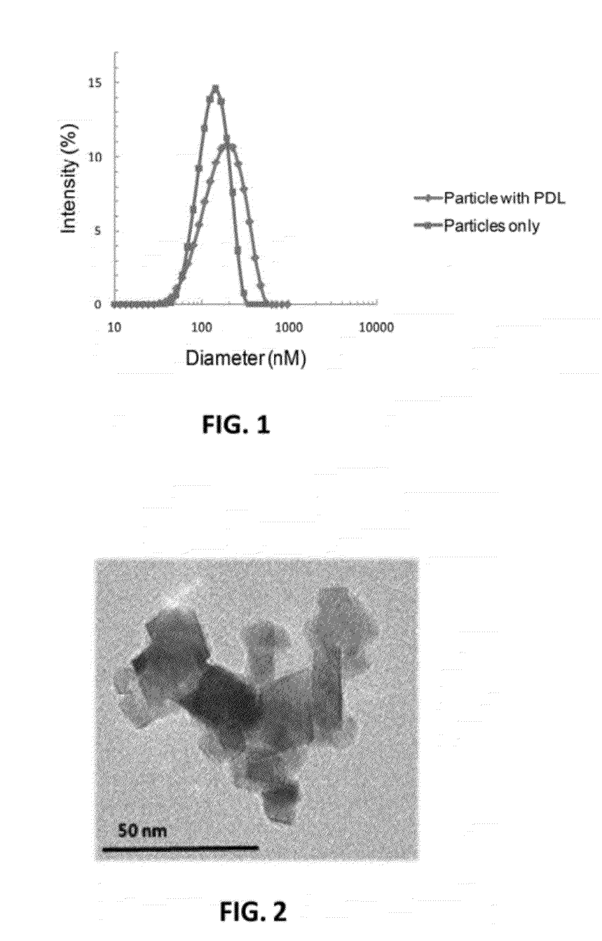

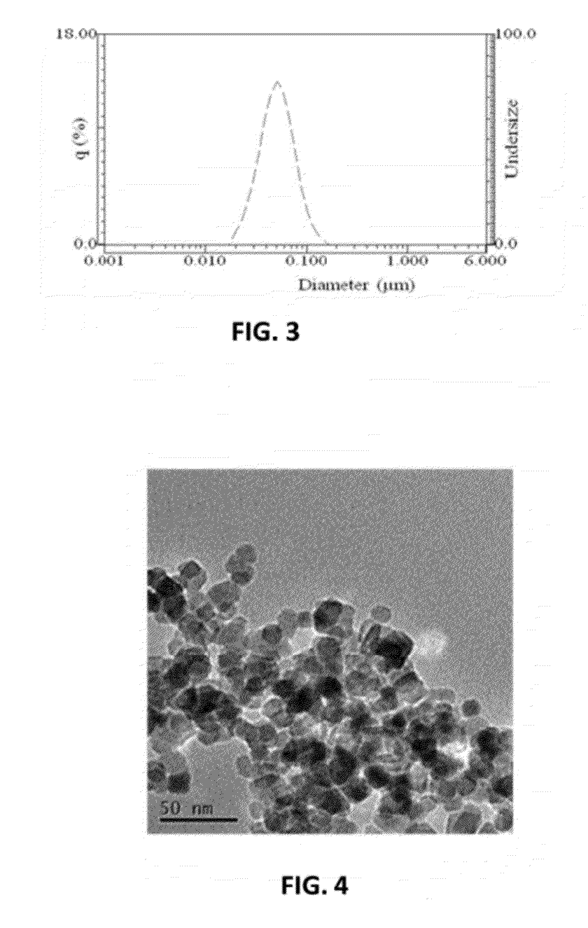

FIG. 1 illustrates a graph of the diameter of the starch-BNF nanoparticles in phosphate buffered saline (PBS) and also with poly D-lysine, a biocompatibilizing agent. FIG. 3 illustrates a graph of the diameter of the dextran-coated nanoparticles stabilized with citrate and suspended in water. Photon correlation spectrograph (PCS) showing mean hydrodynamic diameter of starch coated BNF particles in phosphate buffered saline (PBS) and PBS with poly D-lysine (PDL), a biocompatibilizing agent. Samples were diluted in sterile water to an iron concentration of approximately 0.4 mg/nil prior to analysis.

FIG. 2 illustrates a transmission electron microscopy image from a starch-BNF nanoparticle and FIG. 4 illustrates a transmission electron microscopy image from a dextran-coated nanoparticle. Transmission Electron Microscopy was also used to obtain characteristic images of each of the nanoparticles. Particle solutions were diluted and spin coated onto a carbon film-coated grid to isolate individual particles for imaging. FIGS. 2 and 4 illustrate the imaging of multiple nanoparticle crystals.

The amplitude-dependent SLP for both the starch-BNF and dextran-coated nanoparticles was estimated from measured time-dependent heating in the AMF device, described below, at several applied amplitude (voltage) values from 4 kA/m to 95 kA/m. Sample temperatures were measured with fiber optic probes. The SLP was estimated from the slope, ΔT/Δt of the time-temperature curve using methods described.

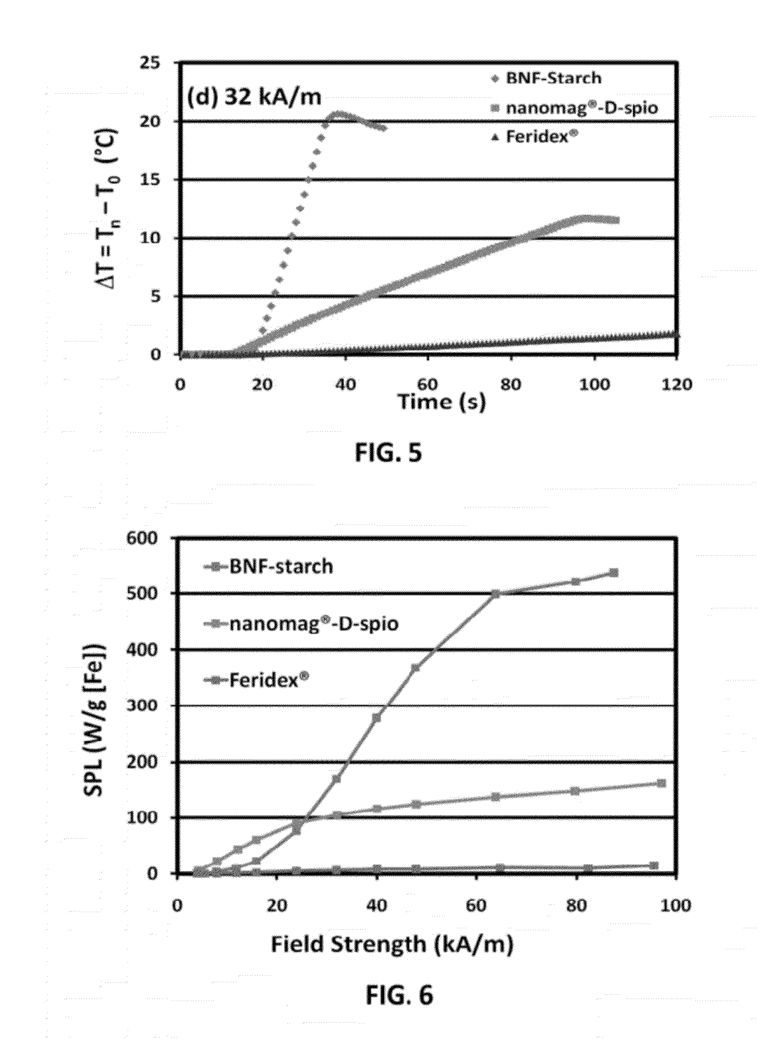

FIG. 5 illustrates a graph of the temperature over time for a sample of starch-BNF nanoparticles activated by AMF. By way of example, a 1-ml volume of a starch-BNF nanoparticle suspension was placed in a standard 12-mm polystyrene test tube and inserted into the insulating sample holder. Equilibrium between the probe, sample, and the calorimeter was confirmed and the AMF power was applied. Temperatures were recorded in 1-s intervals. At each power setting a sample of distilled water was measured to correct for calorimeter heat capacity. The temperature at time interval, Tₙ, was subtracted from the initial temperature, T₀, to yield the net temperature change, ΔTₙ=Tₙ−T₀. Net change water blank temperatures were subtracted from that of the sample to yield the corrected temperature change for each sample.

FIG. 6 illustrates a graph of the heating efficiency for the starch-BNF nanoparticles. The SLP was estimated from the initial and steepest part of the slope, ΔT/Δt , of the time-temperature curve, by fitting a linear weighted least-squares function to the data. The appropriate interval for calculating the slope was determined by analyzing a plot of the incremental temperature change, analogous to the first derivative of the heating rate.

Further, with respect to the contents of the T-C formulation, it may include a chelating agent such ethylenediamine-tetra-acetic acid (EDTA), with a concentration of between 0.01 to 2% w/v to stabilize suspension of the particles in the formulation. However, any other chelating agent known to one of skill in the art could also be used.

The addition of an anti-cancer chemotherapeutic agent such as doxorubicin may be added by first mixing an aqueous solution of doxorubicin with the ethiodized oil/EDTA/particles suspension. Sonication may be used to aid formation of a stable suspension/emulsion. It should also be noted that the T-C formulation is configured such that it can be delivered intra-tumorally or intra-arterially. For instance, the T-C formulation can be administered to a subject using a catheter positioned in an artery adjacent to a tumor selected for treatment. A radiolabeling agent can also be included in the T-C formulation or administered separately. The radiolabeling agent can provide additional contrast for image-guided therapy of the cancer.

Table 1, below, summarizes the specifications of the ethiodized oil-particle mixture:

TABLE 1
Specifications of components of ethiodized oil-magnetic nanoparticle mixture

| | Description | Quantity |
|---|---|---|
| Material | | |
| MION core material | Magnetic iron oxide (γ-Fe₂O₃ | >95% |
| | or Fe₃O₄) | |
| MION core magnetic | Ferromagnetic (ideal) with | Sufficient to provide SLP |
| characteristics | some superparamagnetic | >100 W/g Fe in AMF |
| | character possible | having 20 kA/m @ 150 kHz |
| MION coating | Biocompatible polymer (starch, | <50% w/w of total particle |
| | dextran, poly ethylene glycol, | |
| | etc.) or biocompatible | |
| | surfactant (citric acid, | |
| | phospholipid, polysorbate, | |
| | etc.). | |
| MION Size | 20-150 nm | Mean diameter with |
| | | polydispersity index <0.3 |
| | | as measured in sterile |
| | | water or PBS |
| Iron content of MIONs | 1-100 mg/ml | With particle concentration |
| | | of between about 2 mg/ml |
| | | to 400 mg/ml |
| MION suspending medium | Sterile water or saline | As appropriate to meet |
| | | other criteria |
| Emulsifying agent | Macrocycle chelators (EDTA, | <5% of total formulation, |
| | DOTA, etc.), biocompatible | or lower than limits |
| | surfactants (phospholipids, | allowed by regulatory |
| | etc.), polysorbate | agency |
| Iodine content | 10-420 mg/ml | |
| Tumor-philic carrier fluid | Ethiodized oil | Approximate |
| | | concentrations of |
| | | components |
| Formulation | | |
| MION suspension | As above | 10-50% v/v of |
| | | totalformulation |
| Emulsifying agent | As above | 0-5% v/v of total |
| | | formulation |
| Carrier fluid | As above | 10-50% v/v of total |
| | | formulation |
| Chemotherapeutic agent | Cisplatin, carboplatin, | 0-50% v/v of total |
| | cyclophosphamide, docetaxel, | formulation |
| | doxorubicin, gemcitabine, | |
| | ifosfamide, irinotecan, | |
| | melphalan, mitomycin, | |
| | mitoxantrone, oxaliplatin, | |
| | topotecan, vinorelbine, | |
| | tamoxifen, paclitaxel, etc. | |
| Radioactive isotope | ⁹⁰Y, ¹²⁵I, ¹³¹I, ⁶⁰Co, ¹⁹²Ir, ⁸⁹Sr, | <1 Curie |
| (therapeutic) | ¹⁵³Sm, ¹⁸⁶Re, ^{99m}Tc, etc. | |
| Radioactive isotope | ¹⁸F, ⁶⁴Cu, ¹¹¹In, etc. | <1 mCurie |
| (imaging) | | |

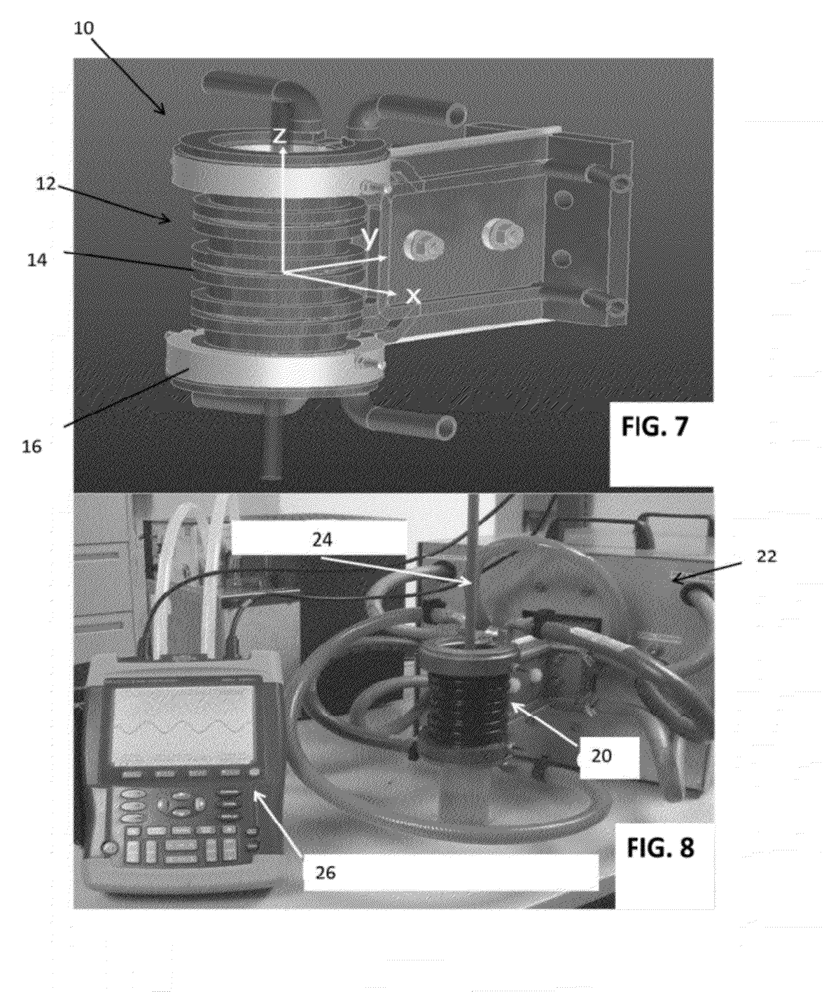

In conjunction with the T-C formulation, an AMF device 10 is used to activate and inductively heat the nanoparticles contained in the T-C formulation. FIG. 7 illustrates a perspective view of an AMF device 10 according to an embodiment of the invention designed to treat small animals. The AMF device includes a modified, four-turn solenoid 12. The solenoid coils 14 can be turned from oxygen-free, high conductivity copper that produces a homogeneous flux density AMF having diameter of 48 mm and 62 mm length. However, any suitable solenoid configuration and/or material known to one of skill in the art can be used. The modified solenoid also comprises high magnetic permeability capping rings 16 to further reduce field inhomogeneities within the cylinder. All braze joints can be made using high temperature copper-silver braze joints.

FIG. 8 illustrates an AMF system according to an embodiment of the invention. As illustrated in FIG. 8, the custom induction coil 20 is mounted to an 4 mega volt-amperes reactive (MVAr) external heat station 22 or matchbox. The external heat station 22 can be a 4 mega volt-amperes reactive (MVAr) external heat station. The external heat station 22 is further connected to industrial induction power 24 by a flexible cable. The industrial induction power can be 80 kW and 135-440 kHz. The system can be tuned to a particular frequency in its range or to account for the impedances of varying output coils by adjusting the capacitance of the matchbox or the power supply as well as by adjusting the series inductor inside the power supply. Field amplitude can be dynamically controlled by adjusting the power supply output voltage. A mounted field probe 24 is disposed within the coil 20, as depicted in FIG. 8. Also, illustrated in FIG. 8, a digital oscilloscope 26 measures the output voltage and frequency of the field probe 24. Together the power supply, external heat station 22, and coil 20 form a resonant circuit. These three components are cooled with a closed-loop, circulating water system maintained between 22° C. and 28° C. The water system provides cooling from a 200-L reservoir with a flow rate of 100 L/min at 900 kPa. It should be noted, that this AMF device and system is described only by way of example, and the AMF device and system can take any form known to one of ordinary skill in the art. Additionally, while this example is used to treat small animals, the device can be expanded for use with larger animals as well as humans.

### EXAMPLES

The following paragraphs describe example experiments performed and results obtained using a T-C formulation and method according to an embodiment of the present invention. However, these examples are not meant to be limiting and the T-C formulation and method can be used in any way known to one of ordinary skill in the art. It should be noted that while the examples below are limited to mice and rabbits, the T-C formulation and method can be expanded to use in other animals and humans as well.

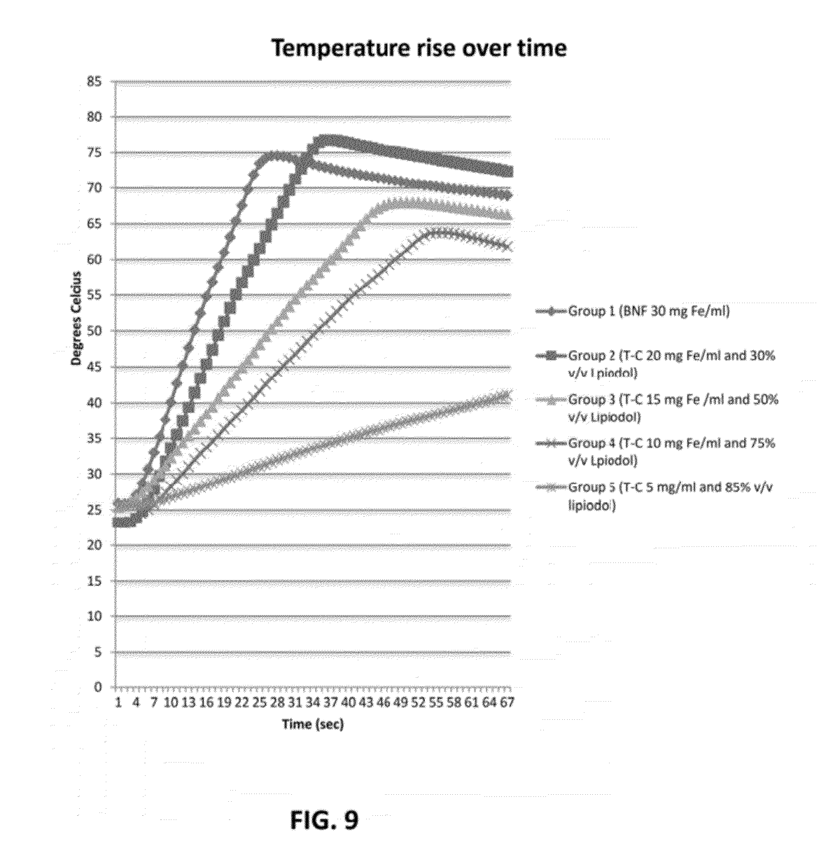

FIG. 9 illustrates a graph depicting the in-vitro heating capacity of four mixtures with a constant volume of 3 ml containing various concentrations of iron and ethiodized oil. The samples were exposed to 150 kHz AC magnetic fields of constant strength in tubes embedded with styrene, for temperature control. The T-C formulation was graded to contain 20, 15, 10, and 5 mg Fe/ml, and 85, 70, 50, and 30% v/v of ethiodized oil, respectively. The Fe took the form of BNF nanoparticles, which are magnetite, starch-coated particles with a high specific absorption rate of 100 Watts/g iron and a mean diameter of 150 nm. These BNF nanoparticles have a concentration of iron ranging between 14 and 30 mg Fe/ml and can produce a temperature increase of 1-3° C./sec depending on the field conditions and nanoparticle concentration in the tumor. The in vitro heating capacity of the above mixtures was compared to the heat generating capacities of the iron nanoparticles alone with a concentration of 30 mg/ml. The polystyrene tubes containing 1 ml of either T-C formulation, BNF particles, ethiodized oil, or phosphate-buffered saline (PBS), were heated until temperatures reached 45° C. in the AMF inductor.

Further, as illustrated in FIG. 9, as the mass (measured in mg) of Fe/ml increased in the four mixtures, the temperature of the T-C formulation increased and also reached a peak temperature faster. The doxorubicin concentration was kept constant (16 mg/ml). A temperature probe was placed inside the tubes and temperatures were recorded at 1-second intervals. The BNF particles alone reached a peak temperature in a shorter amount of time, but that peak temperature was lower than that of the T-C formulation containing 20 mg Fe/ml. The ethiodized oil and PBS did not exhibit heating resulting from the AMF.

In one example experiment using the T-C formulation and method, two male Foxn1nu mice 5 to 8 weeks old and weighing 20.0 and 25.0 g, bearing Hep3B luc flank tumors, as illustrated in FIG. 10, were maintained on a normal diet, ad libitum. Each mouse was anesthetized by injecting 0.2 ml of an anesthesia solution containing ketamine:xylazine. Anesthesia was determined by lack of reflexive response when a hind paw was lightly compressed. After the mouse was anesthetized, two fiber optic temperature probes were placed. One was placed at the center of the tumor by inserting a 16-gauge×1.5 in. hypodermic needle at the center of tumor and threading the fiber optic probe through the needle. The other probe was inserted one cm into the rectum. After the probes were in place, the mouse was wrapped lightly in absorbent paper and inserted into a 50-mL centrifuge tube with the bottom removed.

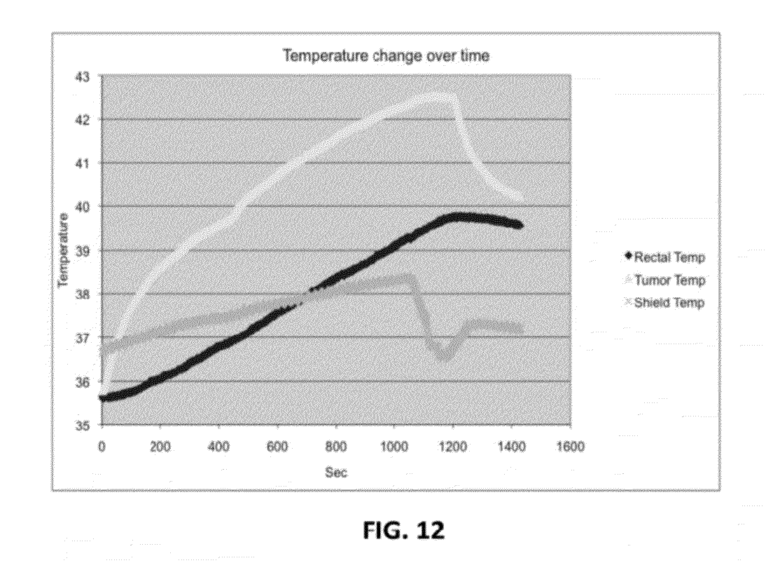

This tube with the mouse was inserted into the felt-lined AMF coil, so that the intended abdominal tumor location of each mouse was positioned in the 1-cm high-amplitude region of the induction coil. Once the mouse was in place and the variables programmed into the controls, the AMF generator was activated. Temperatures were recorded at 1-second intervals for each probe, beginning after each mouse was positioned in the coil and 30 seconds before AMF exposure. FIG. 12 illustrates these temperature readings over time. Intratumoral temperature reached 42 degrees Celsius at 1000 sec from the beginning of experiment. After exposure, each mouse was left in the coil until the core (rectal) temperature began to decrease and all probes were removed. The mouse was removed from the coil and centrifuge tube and placed on a warm recovery pad on its back. When the righting reflex returned, the mouse was returned to its cage. The mice were observed for 48 hours for signs of morbidity. No signs of morbidity were observed during the aforementioned time period.

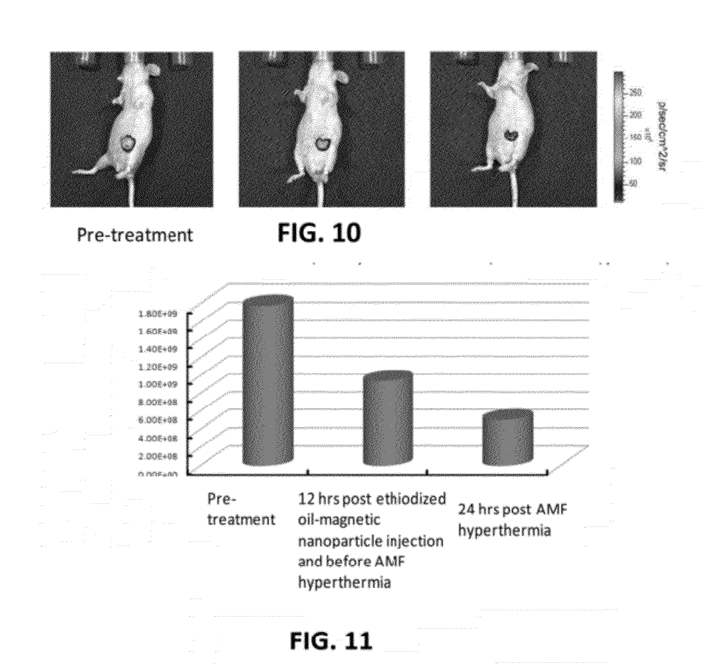

Bioluminescence imaging (BLI) was performed before ethiodized oil-BNF intratumoral injection, at 12 hours following injection and before AMF hyperthermia treatment and at 24 hours after AMF hyperthermia treatment, as illustrated in FIG. 11. FIG. 11 also illustrates that tumor signal decreased significantly over time. Mice were administered D-luciferin at a dose of 150 mg/kg in PBS by i.p. injection, and were anesthetized with 1.5-2% isofluorane for 5-10 min prior to imaging. Animals were then placed onto the warmed stage inside of the IVIS light-tight chamber and anesthesia was maintained with 1.5-2% isofluorane. For the image acquisition, a cooled charge coupled device (CCD) camera was used. Mice were imaged in both the dorsal and ventral positions. Imaging parameters were f/stop 1, bin 4, field of view 12.5 cm, and exposure times ranged from 20 s to 2 min, depending on the strength of the tumor-derived photon emission rates. Signal intensity was quantified as the total photons/s within a uniform region of interest positioned over specific tumor sites, as well as over the entire body, during the data post-processing, with any background bioluminescence subtracted out.

Human hepatocarcinoma cells, Hep3B, that were obtained from a commercial source, were cultured. Thermal sensitivity was determined by standard water bath hyperthermia experiments and cell survival was quantified by clonogenic assay. For AMF exposure 24 hours after seeding, cells were washed with one of the following components: a) T-C formulation (4 concentrations), b) BNF particles, c) ethiodized oil, d) doxorubicin or e) PBS alone, and were AMF heated. Non-heated controls were also tested. A single fiber optic temperature probe was placed inside the plates and temperatures were recorded at 1-second intervals.

In another exemplary experiment, Hep3B cells were implanted by subcutaneous injection of 3×10⁶ Hep3B cells suspended in PBS into the right flank of each mouse that was randomly assigned into 2 main groups (heated and unheated) that were further divided into three subgroups. When tumors reached 100 mm³, these received intratumoral injections of T-C formulation, BNF particles alone, or PBS (3 subgroups). The heated group (9 mice) received AMF hyperthermia. Two mice from each subgroup were euthanized 24 hours following treatment, and the remaining one was euthanized when tumor in the PBS group reached 2000 mm³.

For AMF exposure, each mouse was placed in a chamber constructed from a standard polypropylene 50-mL conical centrifuge tube that was positioned in the center of the solenoid coil. Prior to AMF therapy, each mouse received intraperitoneal injections of ketamine/xylazine (anesthesia) at an approximate dose of a 200-μL injection. Each mouse was placed in the coil for 30 min and exposed to one of the following AMF amplitudes: 0, 24, or 60 kA/m. Because of the nature of the field and coil, each mouse received whole-body AMF exposure. During AMF exposure, mice were placed into the AMF inductor coil and therefore exposed to maximum magnetic field, corresponding to the measured (by field probe) field. Rectal and tumor temperatures were measured at one-second intervals for each mouse exposed to AMF using fiber optic temperature probes.

All mice tolerated well the proposed treatments. We observe a 7-8% degradation of doxorubicin that may be attributed to hyperthermia. Overall, the heating performance of the T-C formulation in mice seems to be at least equal or superior to the BNF particles alone. Moreover, this viscous T-C formulation seems to adopt the tumor seeking, drug- (and iron nanoparticles) carrying properties of ethiodized oil, leading to sustained intratumoral deposition of iron for at least 7 days following treatment. To our knowledge, this unique physiologic behavior of the T-C formulation has not been previously reported and this may prove advantageous over other iron nanoparticle formulations.

In another exemplary experiment using a T-C formulation according to the description and method above, a total of 25 rabbits, implanted with VX2 liver tumors, were randomly assigned into 4 groups for intra-arterial injection of either: a) T-C formulation, b) chemoembolization (C-E), c) BNF particles, or d) PBS. The T-C, BNF and PBS group received AMF hyperthermia. Two animals from the T-C, C-E, PBS groups and one from the BNF group were euthanized at 24 hours. One T-C rabbit was euthanized at 36 hours and the remaining rabbits at 7 days. Tumor samples were processed for hematoxylin and eosin (H&E), TUNEL, Heat Shock Protein 70 (HSP70) and doxorubicin/doxorubicinol (via HPLC-MS) measurements. Nominal bio-distribution of iron in tumors and organs (liver, spleen, lungs) was performed by quantitative iron measurements with ICP-MS and semi-quantitative Prussian Blue (PB) evaluation.

All rabbits tolerated well the proposed treatments. Again, we noticed that the T-C formulation seems to adopt the properties of ethiodized oil, with sustained intratumoral deposition for at least 3 days following treatment. This led to further exploration and successful confirmation that intratumoral iron deposition can be detected at 7 days following treatment. The unique properties of the T-C formulation may serve as a novel platform for testing and developing new therapeutic protocols with repeated hyperthermia treatments without re-injecting iron nanoparticles.

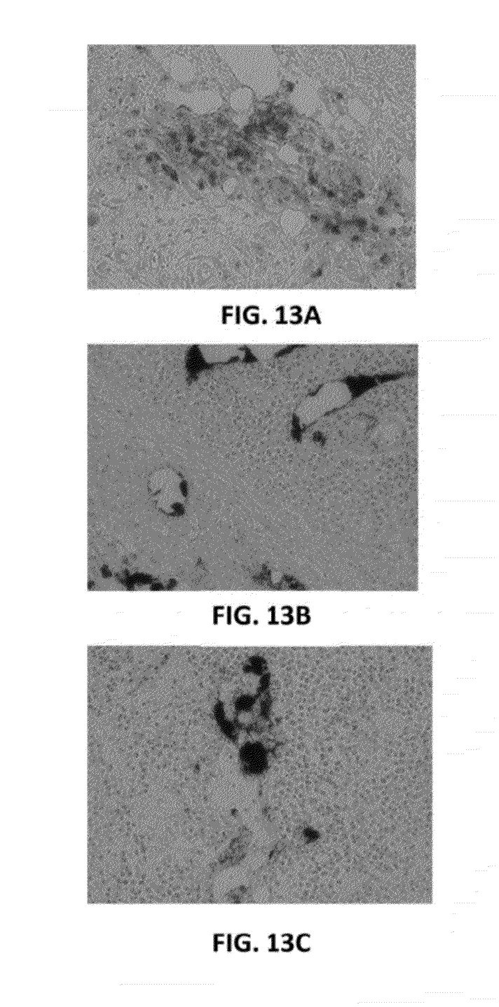

Formalin-fixed tumor, lung, liver, and spleen sections were prepared and stained with hematoxylin and eosin. Perl's reaction was used to qualitatively confirm the presence of ferric (Fe⁺³). Slides were examined by two pathologists and evaluated for necrosis and pigment. Tumor samples were processed for H&E, TUNEL, doxorubicin and doxorubicinol (via HPLC-MS) and heat shock protein 70 (HSP70) measurements. FIGS. 13A-13C illustrates histopathological images of Prussian Blue stained liver VX2 tumor slides at 7 days following treatment for the New Zealand white rabbits. Note that iron is deposited either in the tumor interstitium, as in FIG. 13A or in the tumor vesicles, as in FIGS. 13B and 13C.

Tumor, liver, and spleen was processed for measuring total iron content by inductively-coupled plasma mass spectrometry (ICP-MS) using methods previously described. Each tissue sample was transferred to a 7-mL Teflon microwave digestion vessel, to which 1 mL of optima-grade HNO₃ was added. The vessel was sealed and placed into a 55-mL Teflon microwave digestion vessel, to which 10 mL of ultra-pure H₂0 was added. The 55-mL vessel was sealed and assembled according to the manufacturer's protocol. The assembly was then placed in a microwave, where the tissue samples were digested using the following single-stage ramp-to-temperature microwave method: 15-min ramp to 130° C., with a hold of 30 min.

After cooling, each sample was removed from the microwave and diluted: 35 μL of sample digest and 300 μL of HNO₃ were added to 14.665 mL of ultra-pure H₂O to achieve a final HNO₃ concentration of 2% w/v. Scandium was added to a final concentration of 50 μg/L as an internal standard to monitor instrument drift during analysis. For every batch of 20 tissue samples, 3 samples of Seronorm Trace Elements Whole Blood and 4 reagent blanks (for quality control) were digested and analyzed.

The total iron content of the tissue samples was measured. Each measurement was blank-corrected using the average iron value of the reagent blanks, multiplied by the dilution factor, and adjusted based upon the recovery of iron from Seronorm. An 8-point calibration curve at 0, 1, 5, 10, 50, 100, 500, and 1000 ug/L was obtained. The analytical limit of detection (LOD) was calculated by multiplying the standard deviation of the lowest detectable calibration standard (1 μg/L) by three. For samples with values below the analytical LOD, one-half of the LOD was substituted.

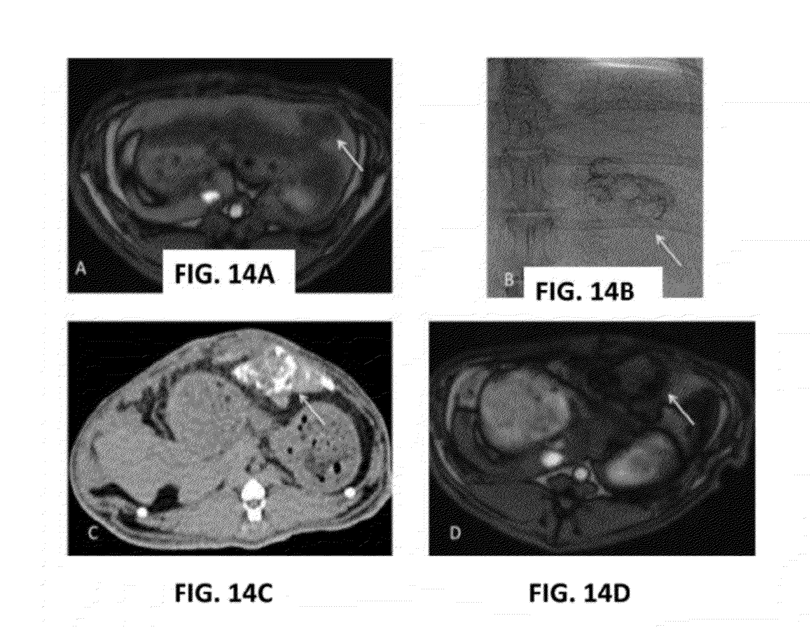

FIGS. 14A-14D show images of the New Zealand white rabbit liver over the experiment. FIG. 14A shows an axial T1 image of liver VX2 tumor in a rabbit before treatment. FIG. 14B illustrates a digital angiographic spot-image of the liver VX2 tumor following intra-arterial injection of the T-C formulation. FIG. 14C illustrates an axial non-contrast enhanced image of the liver and liver VX2 tumor at 7 days after T-C formulation treatment. Note the intratumoral deposition of T-C formulation. FIG. 14D illustrates an axial T1 image of liver VX2 tumor in a rabbit at 7 days after T-C formulation treatment. Note the strong paramagnetic artifact at the tumor site. The arrow on each image indicates the location of the tumor. Overall, all animals were imaged successfully. T-C formulation may serve as a dual imaging marker of therapeutic tumor targeting. CT imaging may serve as the preferred imaging modality for quantifying tumor necrosis, where as MR imaging may serve as the preferred imaging modality for quantifying iron deposition. There seems to be a correlation between iron deposition on MR imaging and histopathology.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

What is claimed is:

1. A thermo-chemoembolization (T-C) formulation for treatment of a tumor in a subject, comprising:
a tumorphilic carrier fluid that selectively accumulates in or near tumor cells to enhance retention of the compound within the tumor, such that the T-C formulation is deliverable either intra-arterially or intratumorally;
a biocompatible suspension of magnetic iron oxide nanoparticles (MIONs) having a magnetic iron oxide core that produces at least 50 Watts of heat per g iron when subjected to an alternating magnetic field having a frequency of between 100 kHz and 1 MHz and a peak-to-peak amplitude of between 5 kA/m and 100 kA/m;
the magnetic iron oxide core is surrounded by a coating; and,
an emulsifying agent.

2. The T-C formulation of claim 1 wherein the tumorphilic carrier fluid comprises ethiodized oil.

3. The T-C formulation of claim 1 wherein the magnetic iron oxide core comprises at least one of the group of γFe₂O₃ and Fe₃O₄.

4. The T-C formulation of claim 3 wherein the magnetic iron oxide core comprises at least one crystal of γFe₂O₃ or Fe₃O₄ or a mixture of γFe₂O₃ and Fe₃O₄

5. The T-C formulation of claim 1 wherein the coating comprises at least one of the group of a biocompatible polymer and a biocompatible surfactant.

6. The T-C formulation of claim 4 wherein the biocompatible polymer comprises at least one of the group of starch, dextran, and polyethylene glycol.

7. The T-C formulation of claim 4 wherein the biocompatible surfactant comprises one of the group of citric acid, phospholipid, and polysorbate.

8. The T-C formulation of claim 1 wherein the emulsifying agent comprises at least one of the group of macrocycle chelators, biocompatible surfactants, and polysorbate.

9. The T-C formulation of claim 8 wherein the macrocycle chelators comprises at least one of the group of EDTA and DOTA.

10. The T-C formulation of claim 1 further comprising an anti-cancer agent comprising at least one of the group of a chemotherapy agent and a radiotherapy agent.

11. The T-C formulation of claim 10 wherein the chemotherapy agent comprises at least one of the group of cisplatin, carboplatin, cyclophosphamide, docetaxel, doxorubicin, gemcitabine, ifosfamide, irinotecan, melphalan, mitomycin, mitoxantrone, oxaliplatin, topotecan, vinorelbine, tamoxifen, and paclitaxol.

12. The T-C formulation of claim 10 wherein the radiotherapy agent comprises at least one of the group of ⁹⁰Y, ¹²⁵I, ¹³¹I, ⁶⁰Co, ¹⁹²Ir, ⁸⁹Sr, ¹⁵³Sm, ¹⁸⁶Re, and ^{99m}Tc.

13. The T-C formulation of claim 1 further comprising a radiolabeling agent.

14. The T-C formulation of claim 13 wherein the radiolabeling agent comprises at least one of the group of ¹⁸F, ⁶⁴Cu, and ¹¹¹In.

15. A method for treating a tumor in a subject comprising:
administering to the subject a tumorphilic thermo-chemoembolization (T-C) formulation comprising a biocompatible suspension of magnetic iron nanoparticles (MIONs) having a magnetic iron oxide core surrounded by a coating;
positioning the subject in an alternating magnetic field (AMF);
applying the AMF to heat the MIONs such that the MIONs increase in temperature;
using image-guided therapy by introducing a radiolabeling agent into the subject; and,
administering an anti-cancer agent to the subject.

16. The method of claim 15 wherein the tumorphilic T-C formulation comprises ethiodized oil.

17. The method of claim 15 wherein the anti-cancer agent comprises at least one of the group of a chemotherapy agent and a radiotherapy agent.

18. The method of claim 15 wherein the magnetic iron oxide core comprises at least one of the group of γFe₂O₃ and Fe₂O₄.

19. The method of claim 15 wherein the coating consists of one of the group of a biocompatible polymer and a biocompatible surfactant.

20. A method for treating a tumor in a subject comprising:
delivering via a catheter to an artery adjacent to a tumor a tumorphilic thermo-chemoembolization (T-C) formulation comprising a biocompatible suspension of magnetic iron nanoparticles (MIONs) having a magnetic iron oxide core surrounded by a coating;
positioning the subject in an alternating magnetic field (AMF);
applying the AMF to heat the MIONs such that the MIONs increase in temperature; and,
administering an anti-cancer agent to the subject.
